(19) United States
(12) Patent Application Publication
(10) Pub. No.: US 2022/0130517 A1
(43) Pub. Date: Apr. 28, 2022

(54) PSYCHOACTIVE TREATMENTS FOR VARIOUS MENTAL HEALTH DISORDERS

(71) Applicant: Ketamine SLC, Salt Lake City, UT (US)

(72) Inventor: Robert C. Hiemstra, Cottonwood Heights, UT (US)

(21) Appl. No.: 17/509,312

(22) Filed: Oct. 25, 2021

Related U.S. Application Data
(60) Provisional application No. 63/104,700, filed on Oct. 23, 2020.

(51) Int. Cl. G16H 20/70; G16H 20/10; G16H 10/20; A61B 5/00; A61K 31/135

(52) U.S. Cl. CPC G16H 20/70; G16H 20/10; G16H 10/20; A61K 31/135; A61B 5/4869; A61B 5/4842; A61B 5/4839

(57) ABSTRACT

Systems and methods for treating a mental health disorder using a psychoactive treatment protocol are described herein. A protocol dosage of the psychoactive chemical such as ketamine may be determined for a patient. A diminished dosage of the psychoactive chemical may be determined for the patient. A dosage progression may be determined based on the diminished dosage and a target dosage. The psychoactive chemical may be administered to the patient according to the dosage progression over a series of therapy sessions. The psychoactive chemical may be administered by intramuscular injection.

### CROSS-REFERENCE TO RELATED APPLICATIONS

The present application claims the benefit of U.S. Provisional Application No. 63/104,700, entitled “SYSTEMS AND METHODS FOR ADMINISTERING A PSYCHOACTIVE TREATMENT FOR VARIOUS MENTAL HEALTH DISORDERS,” filed on Oct. 23, 2020. The entire contents of the above-listed application are hereby incorporated by reference for all purposes.

### BACKGROUND

Many people suffer from one or more mental health disorders. Some research indicates that individuals estimate fifty to ninety percent of their peers suffer from a mental health disorder. The health professions have attempted to address mental health disorders through a combination of drug therapy and counseling. Counseling may be limited as an individual may struggle to implement coping mechanisms and other tools obtained in counseling when carrying the weight of their mental disorder. Drug treatment may be prescribed to supplement counseling so that an individual gets temporary and subtle relief from some of the symptoms of their mental health disorder while learning to implement what is learned in counseling.

### BRIEF DESCRIPTION OF THE DRAWINGS

The present description will be understood more fully when viewed in conjunction with the accompanying drawings of various examples of systems and methods for administering a psychoactive treatment for various mental health disorders. The description is not meant to limit the systems and methods to the specific examples. Rather, the specific examples depicted and described are provided for explanation and understanding of systems and methods for administering a psychoactive treatment for various mental health disorders. Throughout the description, the drawings may be referred to as drawings, figures, and/or FIGS.

FIG. 1 illustrates a project management system, according to an embodiment.

FIG. 2 illustrates a device schematic for various devices used in the project management system of FIG. 1, according to an embodiment.

FIG. 3 illustrates a method of determining a dosage for treating a mental health disorder using a psychoactive chemical, according to an embodiment.

FIG. 4A illustrates a method of administering the psychoactive chemical to a patient to treat the mental health disorder, according to an embodiment.

FIG. 4B illustrates a method of administering ketamine to the patient to treat the mental health disorder, according to an embodiment.

FIG. 5A illustrates a method of evaluating an effectiveness of a target dosage for the psychoactive chemical, according to an embodiment.

FIG. 5B illustrates a method of evaluating an effectiveness of a ketamine dosage, according to an embodiment.

FIG. 6 illustrates a graphical user interface for use in connection with evaluating an outcome of a patient in response to a treatment for a mental health disorder, according to an embodiment.

FIG. 7 illustrates a method of evaluating an outcome of a patient in response to a treatment for a mental health disorder, according to an embodiment.

FIG. 8 illustrates a block diagram of a treatment environment for treating a patient having a mental health disorder using ketamine, according to an embodiment.

### DETAILED DESCRIPTION

Systems and methods for administering psychoactive treatments for various mental health disorders as disclosed herein will become better understood through a review of the following detailed description in conjunction with the figures. The detailed description and figures provide merely examples of the various embodiments of systems and methods for administering psychoactive treatments for various mental health disorders. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

Conventional treatment for a mental health disorder may include prescribing a selective serotonin reuptake inhibitor (SSRI), serotonin and norepinephrine reuptake inhibitor (SNRI), tricyclic antidepressant (TCA), monoamine oxidase inhibitor (MAOI), and/or noradrenaline and specific serotoninergic antidepressant (NASSA). Typically, such drugs may be prescribed alongside a form of counseling such as cognitive behavior therapy. However, such drugs have been shown to have limited effectiveness in treating mild to moderate mental health disorders such as depression, anxiety, post-traumatic stress disorder (PTSD), and so forth. Many individuals with mild to moderate mental health disorders for whom drug and counseling therapy have not worked simply live under the weight of their condition. Such individuals may be driven to destructive behaviors such as addiction, suicide, and/or destructive habits in relationships as a result of unresolved mental health disorders.

One alternative to the above-mentioned treatments that have been tested is administration of a dissociative drug such as ketamine. Typical treatments include intravenous (IV)-administration of ketamine in six separate sessions over twelve days, with two days between each session. The dosage during each session may be calculated according to the patient's body mass, where a single dose is calculated as 0.5 milligrams (mg) of ketamine per kilogram (kg) of body mass of the patient. The dose is administered via IV drip over 40 minutes. Results of such tests have indicated some patients experience relief from the symptoms of their mental health disorder for a period of time including weeks and/or months. Unfortunately, the current ketamine regimens for treating mental health disorders suffer from several drawbacks. The regimens can be cost-prohibitive, costing up to $1,500 per month in some cases. The regimens require the patient to set aside six days for receiving the treatment for every effective period of the treatment, which for many patients can be discouraging. Also, whereas ketamine may have some emergence (e.g. psychedelic) effects, a patient receiving an IV drip who experiences an emergence event may inadvertently pull out the IV, possibly injuring themselves and damaging equipment. Furthermore, because the ketamine is administered via IV, the setting for the treatment may be clinical and sterile, which may undermine treatment of the patient's mental health disorder.

Regardless of the treatment technique used to treat a mental health disorder, evaluating patient outcomes after treatment can be challenging. For example, physicians and other healthcare practitioners traditionally use the metric of a 50% reduction in patient symptoms as indicating that a treatment has been successful. However, symptoms of mental health disorders can be difficult to quantify, as can reductions or changes in those symptoms. Furthermore, changes in symptoms may not be permanent. Symptoms may return or worsen later even if they are improved immediately following treatment. Thus, a treatment to which a patient responds well but only for a short time may be seen as less effective than a treatment that results in a longer interval of improvement or freedom from symptoms for the patient.

Various implementations of this disclosure address the shortcomings of current treatments described above. A system of treating a mental health disorder using a psychoactive chemical may include a serene and comfortable setting that imitates a natural environment, a syringe configured for intramuscular injection, and a dose of the psychoactive chemical, such as a dose of ketamine. A method of determining a dosage for the psychoactive chemical may include calculating a protocol dosage for a patient, determining a diminished protocol dosage for the patient, determining a dosage progression for the patient based on a target dosage for the patient, and administering doses of the psychoactive chemical to the patient according to the diminished protocol dosage and dosage progression. The doses may be administered via intramuscular injection using the syringe. The dosage progression (termed “steps”) may include increasing the dose of the psychoactive chemical given to the patient over a series of sessions and sub-sessions (termed “legs” of a session) until the target dosage is met. Subsequently, the target dosage may be administered at the end of an effective period of the treatment.

The various implementations of this disclosure may improve on the previous treatments. For example, the steps may allow the patient's body to adjust to what may be a new drug the patient has never taken before, which in turn may reduce the probability and/or severity of an emergence event. The serene and peaceful environment may reduce and/or eliminate the likelihood the patient will experience negative feelings and/or emotions during the emergence event. The intramuscular injection may reduce the risk of injury and/or damage in the event the patient experiences an emergence event. Stepping the dosage according to the techniques described in this disclosure may enable the patient to take higher doses during a leg of a session without the patient experiencing an emergence event. Because the patient is able to take higher doses, the patient may only need one session at the end of the effective period as opposed to six sessions under previous treatment regimens. The stepped dosage may also reduce the cost to the patient, where the patient may need to take less than what would be needed under previous treatment regimens and/or the patient may spend less time in the clinic (e.g. may reduce the cost of clinic time versus previous solutions requiring six visits). Thus, the various implementations of this disclosure represent a marked improvement to the treatment of mental health disorders using a psychoactive chemical.

Aspects of this disclosure can also improve measurement and evaluation of patient outcomes following a treatment protocol for a mental health disorder. Because of the intangible nature of some mental health disorder symptoms and the variability of those symptoms over time, it can be difficult to objectively classify a treatment as successful of unsuccessful. A standardized health questionnaire can be given to patients. The questionnaire can include questions relating to the mental health disorder of the patient. Patient responses to the questionnaire can be received, and a score can be generated based on the responses. The score can be used to determine whether the patient is a candidate for a particular treatment protocol. Following treatment, the patient can again respond to the questionnaire, and the patient's responses can be scored to determine whether the treatment was effective in eliminating or improving the symptoms of the patient. In addition, the patient may be asked to respond to the questionnaire periodically after the treatment. Scoring the patient's responses to the questionnaire over time can allow for identifying a duration of a time interval during which the patient's symptoms remain improved as a result of the treatment.

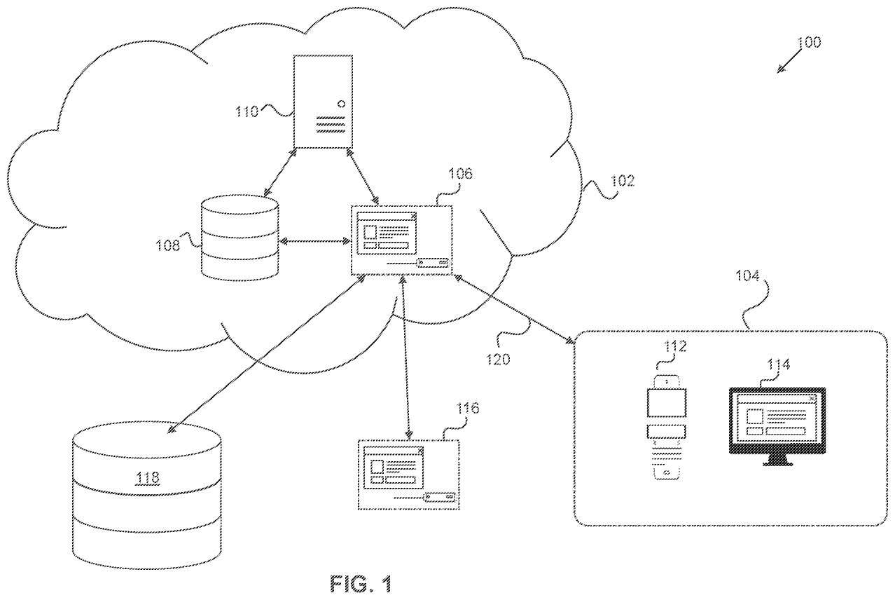

FIG. 1 illustrates a project management system 100, according to an embodiment. The project management system 100 includes internal and external data resources for managing a project. The project management system 100 may result in reduced memory allocation at client devices and may conserve memory resources for application servers.

The project management system 100 may include a cloud-based data management system 102 and a user device 104. The cloud-based data management system 102 may include an application server 106, a database 108, and a data server 110. The user device 104 may include one or more devices associated with user profiles of the project management system 100, such as a smartphone 112 and/or a personal computer 114. The project management system 100 may include external resources such as an external application server 116 and/or an external database 118. The various elements of the project management system 100 may communicate via various communication links 120. An external resource may generally be considered a data resource owned and/or operated by an entity other than an entity that utilizes the cloud-based data management system 102 and/or the user device 104.

The project management system 100 may be web-based. The user device 104 may access the cloud-based data management system 102 via an online portal set up and/or managed by the application server 106. The project management system 100 may be implemented using a public Internet. The project management system 100 may be implemented using a private intranet. Elements of the project management system 100, such as the database 108 and/or the data server 110, may be physically housed at a location remote from an entity that owns and/or operates the project management system 100. For example, various elements of the project management system 100 may be physically housed at a public service provider such as a web services provider. Elements of the project management system 100 may be physically housed at a private location, such as at a location occupied by the entity that owns and/or operates the project management system 100.

The communication links 120 may be direct or indirect. A direct link may include a link between two devices where information is communicated from one device to the other without passing through an intermediary. For example, the direct link may include a Bluetooth™ connection, a Zigbee® connection, a Wifi Direct™ connection, a near-field communications (NFC) connection, an infrared connection, a wired universal serial bus (USB) connection, an ethernet cable connection, a fiber-optic connection, a firewire connection, a microwire connection, and so forth. In another example, the direct link may include a cable on a bus network. “Direct,” when used regarding the communication links 120, may refer to any of the aforementioned direct communication links.

An indirect link may include a link between two or more devices where data may pass through an intermediary, such as a router, before being received by an intended recipient of the data. For example, the indirect link may include a wireless fidelity (WiFi) connection where data is passed through a WiFi router, a cellular network connection where data is passed through a cellular network router, a wired network connection where devices are interconnected through hubs and/or routers, and so forth. The cellular network connection may be implemented according to one or more cellular network standards, including the global system for mobile communications (GSM) standard, a code division multiple access (CDMA) standard such as the universal mobile telecommunications standard, an orthogonal frequency division multiple access (OFDMA) standard such as the long term evolution (LTE) standard, and so forth. “Indirect,” when used regarding the communication links 120, may refer to any of the aforementioned indirect communication links.

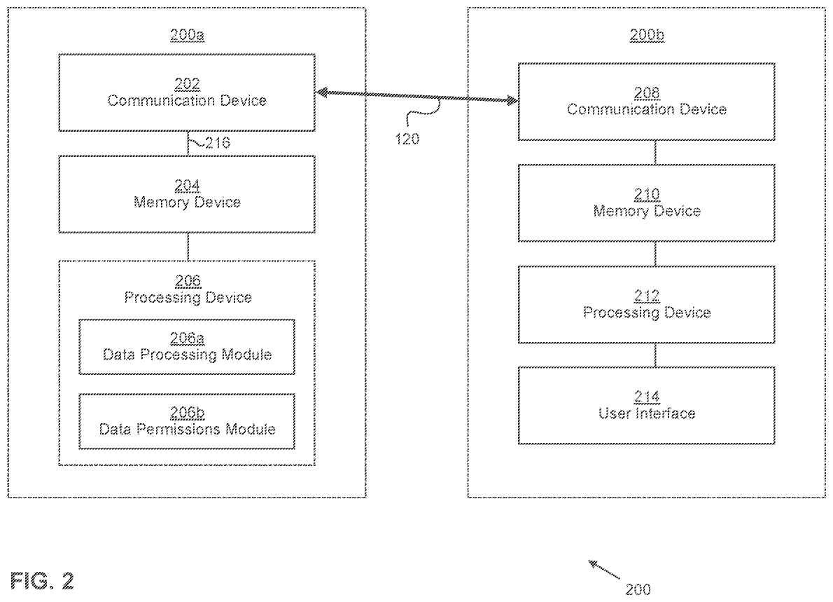

FIG. 2 illustrates a device schematic 200 for various devices used in the project management system 100, according to an embodiment. A server device 200a may moderate data communicated to a client device 200b based on data permissions to minimize memory resource allocation at the client device 200b.

The server device 200a may include a communication device 202, a memory device 204, and a processing device 206. The processing device 206 may include a data processing module 206a and a data permissions module 206b, where module refers to specific programming that governs how data is handled by the processing device 206. The client device 200b may include a communication device 208, a memory device 210, a processing device 212, and a user interface 214. Various hardware elements within the server device 200a and/or the client device 200b may be interconnected via a system bus 216. The system bus 216 may be and/or include a control bus, a data bus, and address bus, and so forth. The communication device 202 of the server device 200a may communicate with the communication device 208 of the client device 200b.

The data processing module 206a may handle inputs from the client device 200a. The data processing module 206a may cause data to be written and stored in the memory device 204 based on the inputs from the client device 200b. The data processing module 206a may retrieve data stored in the memory device 204 and output the data to the client device 200a via the communication device 202. The data permissions module 206b may determine, based on permissions data stored in the memory device, what data to output to the client device 200b and what format to output the data in (e.g. as a static variable, as a dynamic variable, and so forth). For example, a variable that is disabled for a particular user profile may be output as static. When the variable is enabled for the particular user profile, the variable may be output as dynamic.

The server device 200a may be representative of the cloud-based data management system 102. The server device 200a may be representative of the application server 106. The server device 200a may be representative of the data server 110. The server device 200a may be representative of the external application server 116. The memory device 204 may be representative of the database 108 and the processing device 206 may be representative of the data server 110. The memory device 204 may be representative of the external database 118 and the processing device 206 may be representative of the external application server 116. For example, the database 108 and/or the external database 118 may be implemented as a block of memory in the memory device 204. The memory device 204 may further store instructions that, when executed by the processing device 206, perform various functions with the data stored in the database 108 and/or the external database 118.

Similarly, the client device 200b may be representative of the user device 104. The client device 200b may be representative of the smartphone 112. The client device 200b may be representative of the personal computer 114. The memory device 210 may store application instructions that, when executed by the processing device 212, cause the client device 200b to perform various functions associated with the instructions, such as retrieving data, processing data, receiving input, processing input, transmitting data, and so forth.

As stated above, the server device 200a and the client device 200b may be representative of various devices of the project management system 100. Various of the elements of the project management system 100 may include data storage and/or processing capabilities. Such capabilities may be rendered by various electronics for processing and/or storing electronic signals. One or more of the devices in the project management system 100 may include a processing device. For example, the cloud-based data management system 102, the user device 104, the smartphone 112, the personal computer 114, the external application server 116, and/or the external database 118 may include a processing device. One or more of the devices in the project management system 100 may include a memory device. For example, the cloud-based data management system 102, the user device 104, the smartphone 112, the personal computer 114, the external application server 116, and/or the external database 118 may include the memory device.

The processing device may have volatile and/or persistent memory. The memory device may have volatile and/or persistent memory. The processing device may have volatile memory and the memory device may have persistent memory. Memory in the processing device may be allocated dynamically according to variables, variable states, static objects, and permissions associated with objects and variables in the project management system 100. Such memory allocation may be based on instructions stored in the memory device. Memory resources at a specific device may be conserved relative to other systems that do not associate variables and other object with permission data for the specific device.

The processing device may generate an output based on an input. For example, the processing device may receive an electronic and/or digital signal. The processing device may read the signal and perform one or more tasks with the signal, such as performing various functions with data in response to input received by the processing device. The processing device may read from the memory device information needed to perform the functions. For example, the processing device may update a variable from static to dynamic based on a received input and a rule stored as data on the memory device. The processing device may send an output signal to the memory device, and the memory device may store data according to the signal output by the processing device.

The processing device may be and/or include a processor, a microprocessor, a computer processing unit (CPU), a graphics processing unit (GPU), a neural processing unit, a physics processing unit, a digital signal processor, an image signal processor, a synergistic processing element, a field-programmable gate array (FPGA), a sound chip, a multi-core processor, and so forth. As used herein, “processor,” “processing component,” “processing device,” and/or “processing unit” may be used generically to refer to any or all of the aforementioned specific devices, elements, and/or features of the processing device.

The memory device may be and/or include a computer processing unit register, a cache memory, a magnetic disk, an optical disk, a solid-state drive, and so forth. The memory device may be configured with random access memory (RAM), read-only memory (ROM), static RAM, dynamic RAM, masked ROM, programmable ROM, erasable and programmable ROM, electrically erasable and programmable ROM, and so forth. As used herein, “memory,” “memory component,” “memory device,” and/or “memory unit” may be used generically to refer to any or all of the aforementioned specific devices, elements, and/or features of the memory device.

Various of the devices in the project management system 100 may include data communication capabilities. Such capabilities may be rendered by various electronics for transmitting and/or receiving electronic and/or electromagnetic signals. One or more of the devices in the project management system 100 may include a communication device, e.g., the communication device 202 and/or the communication device 208. For example, the cloud-based data management system 102, the user device 104, the smartphone 112, the personal computer 114, the application server 116, and/or the external database 118 may include a communication device.

The communication device may include, for example, a networking chip, one or more antennas, and/or one or more communication ports. The communication device may generate radio frequency (RF) signals and transmit the RF signals via one or more of the antennas. The communication device may receive and/or translate the RF signals. The communication device may transceive the RF signals. The RF signals may be broadcast and/or received by the antennas.

The communication device may generate electronic signals and transmit the RF signals via one or more of the communication ports. The communication device may receive the RF signals from one or more of the communication ports. The electronic signals may be transmitted to and/or from a communication hardline by the communication ports. The communication device may generate optical signals and transmit the optical signals to one or more of the communication ports. The communication device may receive the optical signals and/or may generate one or more digital signals based on the optical signals. The optical signals may be transmitted to and/or received from a communication hardline by the communication port, and/or the optical signals may be transmitted and/or received across open space by the networking device.

The communication device may include hardware and/or software for generating and communicating signals over a direct and/or indirect network communication link. For example, the communication component may include a USB port and a USB wire, and/or an RF antenna with Bluetooth™ programming installed on a processor, such as the processing component, coupled to the antenna. In another example, the communication component may include an RF antenna and programming installed on a processor, such as the processing device, for communicating over a Wifi and/or cellular network. As used herein, “communication device” “communication component,” and/or “communication unit” may be used generically herein to refer to any or all of the aforementioned elements and/or features of the communication component.

Various of the elements in the project management system 100 may be referred to as a “server.” Such elements may include a server device. The server device may include a physical server and/or a virtual server. For example, the server device may include one or more bare-metal servers. The bare-metal servers may be single-tenant servers or multiple tenant servers. In another example, the server device may include a bare metal server partitioned into two or more virtual servers. The virtual servers may include separate operating systems and/or applications from each other. In yet another example, the server device may include a virtual server distributed on a cluster of networked physical servers. The virtual servers may include an operating system and/or one or more applications installed on the virtual server and distributed across the cluster of networked physical servers. In yet another example, the server device may include more than one virtual server distributed across a cluster of networked physical servers.

The term server may refer to functionality of a device and/or an application operating on a device. For example, an application server may be programming instantiated in an operating system installed on a memory device and run by a processing device. The application server may include instructions for receiving, retrieving, storing, outputting, and/or processing data. A processing server may be programming instantiated in an operating system that receives data, applies rules to data, makes inferences about the data, and so forth. Servers referred to separately herein, such as an application server, a processing server, a collaboration server, a scheduling server, and so forth may be instantiated in the same operating system and/or on the same server device. Separate servers may be instantiated in the same application or in different applications.

Various aspects of the systems described herein may be referred to as “data.” Data may be used to refer generically to modes of storing and/or conveying information. Accordingly, data may refer to textual entries in a table of a database. Data may refer to alphanumeric characters stored in a database. Data may refer to machine-readable code. Data may refer to images. Data may refer to audio. Data may refer to, more broadly, a sequence of one or more symbols. The symbols may be binary. Data may refer to a machine state that is computer-readable. Data may refer to human-readable text.

Various of the devices in the project management system 100, including the server device 200a and/or the client device 200b, may include a user interface for outputting information in a format perceptible by a user and receiving input from the user, e.g., the user interface 214. The user interface may include a display screen such as a light-emitting diode (LED) display, an organic LED (OLED) display, an active-matrix OLED (AMOLED) display, a liquid crystal display (LCD), a thin-film transistor (TFT) LCD, a plasma display, a quantum dot (QLED) display, and so forth. The user interface may include an acoustic element such as a speaker, a microphone, and so forth. The user interface may include a button, a switch, a keyboard, a touch-sensitive surface, a touchscreen, a camera, a fingerprint scanner, and so forth. The touchscreen may include a resistive touchscreen, a capacitive touchscreen, and so forth.

Various methods are described below. The methods may be implemented by the data analysis system 100 and/or various elements of the data analysis system described above. For example, inputs indicated as being received in a method may be input at the client device 200b and/or received at the server device 200a. Determinations made in the methods may be outputs generated by the processing device 206 based on inputs stored in the memory device 204. Correlations performed in the methods may be executed by the correlation module 206a. Inference outputs may be generated by the inference module 206b. Key data and/or actionable data may be stored in the knowledge database 204b. Correlations between key data and actionable data may be stored in the knowledge database 204b. Outputs generated in the methods may be output to the output database 204c and/or the client device 200b. In general, data described in the methods may be stored and/or processed by various elements of the data analysis system 100.

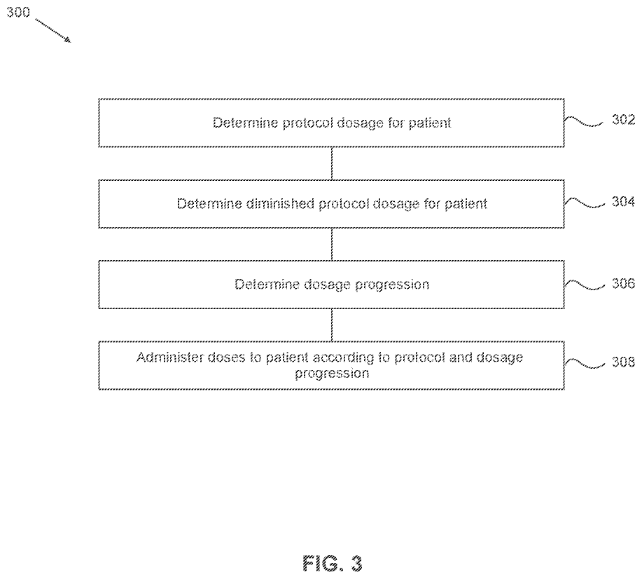

FIG. 3 illustrates a method 300 of determining a dosage for treating a mental health disorder using a psychoactive chemical, according to an embodiment. Some of the features in FIG. 3 may be the same as or similar to some of the features in the other FIGS. described herein as noted by same and/or similar reference characters, unless expressly described otherwise. Additionally, reference may be made to features shown in any of the other FIGS. described herein and not shown in FIG. 3. Elements of the method 300 may be executed in one or more ways such as by a human, by a processing device, by mechanisms operating automatically or under human control, and so forth.

The method 300 may include determining a protocol dosage for the patient (block 302). The protocol dosage may be calculated from an established guideline for using the psychoactive chemical (e.g. ketamine). The guideline may be established by a government regulatory authority, professional regulatory authority, by research conducted using the psychoactive chemical, and so forth. In one example, the guideline may be established by a patient's subjective sense of dissociation. For example, a protocol dosage for ketamine may range from 0.3 mg of ketamine per kg of patient body mass to 1.0 mg/kg. The protocol dosage for ketamine may be 0.3, 0.35, 0.4, 0.45, 0.5, 0.55, 0.6, 0.65, 0.7, 0.75, 0.8, 0.85, 0.9, or 0.95 mg/kg. The protocol dosage may include a time period over which the protocol dosage is administered. The protocol dosage may call for the protocol dosage to be administered all at once (e.g. over 1-2 seconds). The protocol dosage may call for the dose to be administered gradually, such as over a period of 5-10 seconds or a period of minutes.

The method 300 may include determining a diminished protocol dosage for the patient (block 304). The diminished protocol dosage may be less than the protocol dosage. The diminished protocol dosage may be based on the patient's mass. For example, a patient having less than 80 kg of body mass may have a diminished protocol dosage that is 10 mg less than the protocol dosage. A patient having 80 kg or more of body mass may have a diminished protocol dosage that is 5 mg less than the protocol dosage. The protocol dosage may be reduced according to an amount scaled with the patient's body mass. The protocol dosage may, for example, be reduced from between 5 mg to 10 mg, where a higher body mass correlates with a lesser reduction. Reducing the protocol dosage may minimize the likelihood of an emergence event and/or enable the patient to acclimate to the psychoactive chemical.

The method 300 may include determining a dosage progression for the patient (block 306). The dosage progression may designate an amount of increase in the dose provided to and/or taken by the patient at each step of the treatment regimen. The increase may be based on the patient's body mass. For example, a patient having less than 80 kg of body mass may have a prescribed increase of 5 mg per step. A patient having 80 kg or more of body mass may have a prescribed increase of 10 mg per step. In other embodiments, a patient having a body weight less than 140 pounds may have a prescribed increase of 5 mg per step, while a patient having a body weight of 140 pounds or more may have a prescribed increase of 10 mg per step. The dosage progression may be scaled according to the patient's body mass, where, as body mass increases, the prescribed increase increases. Increasing the protocol dosage at each step may enable the patient to continue realizing therapeutic effects of the psychoactive chemical while the patient's body acclimates to the psychoactive chemical. All the while the patient may avoid experiencing emergence events.

The method 300 may include administering doses of the psychoactive chemical to the patient according to the diminished protocol dosage and the dosage progression (block 308). The doses may be administered via intramuscular injection. The doses may be administered via sublingual or oral consumption. The doses may be administered via IV. The doses may be administered via a nasal spray. The doses may be administered via a troche. The doses may be administered in a serene and peaceful setting. The setting may imitate a natural setting (e.g. specific ecological environment) in color, decor, lighting, ambient sound, and so forth. A treatment ally may be present in the setting to help the patient navigate the feelings and emotions the patient experiences during an active period after the patient receives a dose.

The patient may receive the treatment over a period of time. The period of time may be segmented into sessions and/or sub-sessions. A session may include administration of one or more doses. For example, a session may include administration of two doses. A session may include administration of three doses. A session may include administration of four doses. A dose provided during a session may be referred to as a sub-session or leg of the session. The treatment regimen may include 13 steps administered over six sessions. The treatment regimen may include follow-up sessions that include one leg, two legs, three legs, and so forth. There may be a waiting period between administrations of doses during a single session. Such a waiting period can be referred to as an inter-dosage period. A duration of the inter-dosage period can be selected based on a half-life of the psychoactive chemical to be administered, so that the patient is no longer feeling an effect from a previous dosage at the time when a subsequent dosage is administered. For example, when ketamine is the psychoactive chemical, the inter-dosage period can be 30 minutes. There may be a waiting period between successive sessions. Such a waiting period can be referred to as an inter-session period.

An example treatment regimen using ketamine may include interviewing a patient to determine the patient's symptoms and to determine whether the patient is a candidate for treatment using a psychoactive chemical. Once the patient is determined to qualify for treatment, the patient may be weighed. For example, the patient may weigh 177 pounds, which may indicate the patient has a body mass of approximately 80 kg. In this example, the protocol dosage may be determined to be 40 mg, corresponding to 0.5 mg per kg of patient body mass. The diminished protocol dosage may be determined to be 5 mg, and the dose increase of the dosage progression may be determined to be 10 mg for the first eight steps, 5 mg for the ninth step, 10 mg for the tenth step, and 0 mg for subsequent steps. The steps may be planned according to the diminished protocol dosage and the dosage progression, where: the first step includes administering 35 mg of ketamine via intramuscular injection; the second step includes administering 45 mg of ketamine via intramuscular injection; the third step includes administering 55 mg of ketamine via intramuscular injection; the fourth step includes administering 65 mg of ketamine via intramuscular injection; the fifth step includes administering 75 mg of ketamine via intramuscular injection; the sixth step includes administering 85 mg of ketamine via intramuscular injection; the seventh step includes administering 95 mg of ketamine via intramuscular injection; the eighth step includes administering 105 mg of ketamine via intramuscular injection; the ninth step includes administering 110 mg of ketamine via intramuscular injection; the tenth step includes administering 120 mg of ketamine via intramuscular injection; and subsequent steps include administering 120 mg of ketamine via intramuscular injection.

The first and second steps may be administered during two consecutive legs of a first session of the treatment regimen. A leg may be 20 to 40 minutes long. A leg may be 30 minutes long. The third and fourth steps may be administered during two consecutive legs of a second session. The fifth and sixth steps may be administered during two consecutive legs of a third session. The seventh and eighth steps may be administered during two consecutive legs of a fourth session. The ninth and tenth steps may be administered during two consecutive legs of a fifth session. Subsequent steps may be administered during consecutive legs of a sixth session.

Subsequent sessions may be spaced by and inter-session period having a duration in the range of 20-52 hours. Inter-session periods of a treatment regimen may all be of equal duration. Some inter-session periods of a treatment regimen may be of different duration. For example, the six sessions may occur over six days, where the second session starts 24 hours after the first session started, the third session starts 24 hours after the second session started, and so forth. The six sessions may occur over twelve days, where the second session starts 48 hours after the first session started, the third session starts 48 hours after the second session started, and so forth. Sessions may be evenly spaced or unevenly spaced. For example, the second session may start 12 hours after the first session started. The third session may start 18 hours after the second session started. The fourth session may start 24 hours after the third session started. The fifth and sixth session may start at 24-hour intervals as well. As discussed herein, as the dosage is administered throughout the intervals, the treatment regimen may be switched to or include the use of troches.

The progressed dosage may have an objective of increasing the diminished dosage to a target dosage. The target dosage may be a dosage level at which the patient may receive a sufficient amount of the psychoactive chemical during a single session to render the patient symptom-free for a threshold period. The threshold period may, for example, be three months. As an example, the diminished dosage may be ketamine at 0.4 mg/kg and the target dosage may be 1.2 mg/kg. At 1.2 mg/kg, the patient may receive enough ketamine during one session to render the patient symptom-free for approximately three months. Administering the diminished dosage during the first session and then progressing the dosage up to the target dosage may minimize any potential adverse effect the ketamine may have on the patient, such as headaches, nausea, dizziness, and/or emergence events. In addition, other medications can also be administered before, during, or after any session to reduce or eliminate side effects cause by the psychoactive chemical. For example, any combination of an anti-nauseant medication, an anti-hypertensive medication, and an anti-anxiety medication can be administered to the patient at any time throughout the treatment regimen.

The method 300 can also include waiting for a threshold period during which the patient's symptoms are improved after the sixth therapy session. In response to the patient's symptoms returning after the threshold period, a seventh therapy session can be performed. The seventh therapy session can include administering three booster dosages. Administration of each booster dosage can be separated by a seventh inter-dosage period. In some embodiments, an amount of each booster dosage can be selected to be equal to the target dosage.

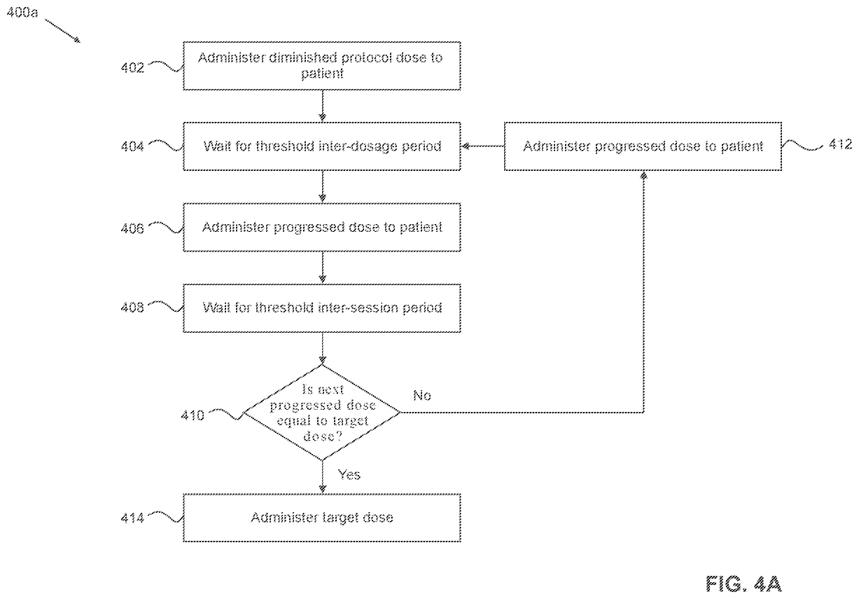

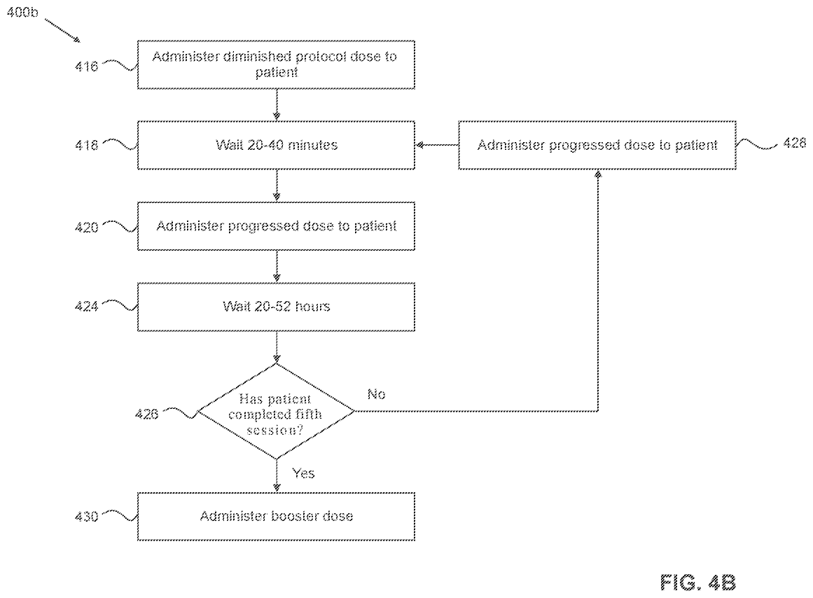

FIG. 4A illustrates a method 400a of administering the psychoactive chemical to a patient to treat the mental health disorder, according to an embodiment. Some of the features in FIG. 4A may be the same as or similar to some of the features in the other FIGS. described herein as noted by same and/or similar reference characters, unless expressly described otherwise. Additionally, reference may be made to features shown in any of the other FIGS. described herein and not shown in FIG. 4A. Elements of the method 400a may be executed in one or more ways such as by a human, by a processing device, by mechanisms operating automatically or under human control, and so forth.

The method 400a may be a dosage progression method. The method 400a may include administering the diminished protocol dose to the patient (block 402). The method 400a may include waiting for a threshold inter-dosage period (block 404). The method 400a may include administering a progressed does to the patient (block 406). The method 400a may include waiting for a threshold inter-session period (block 408). The method 400a may include determining whether the next progressed dosage is equal to the target dosage (block 410). If not, the next progressed dose may be administered to the patient (block 412). If so, the target dose may be administered to the patient (block 414).

FIG. 4B illustrates a method 400b of administering ketamine to a patient to treat the mental health disorder, according to an embodiment. Some of the features in FIG. 4A may be the same as or similar to some of the features in the other FIGS. described herein as noted by same and/or similar reference characters, unless expressly described otherwise. Additionally, reference may be made to features shown in any of the other FIGS. described herein and not shown in FIG. 4A. Elements of the method 400b may be executed in one or more ways such as by a human, by a processing device, by mechanisms operating automatically or under human control, and so forth.

The method 400b may be a dosage progression for a patient receiving ketamine. The method 400b may include administering the diminished protocol dose of ketamine to the patient (block 416). The method 400b may include waiting for twenty to forty minutes (block 418). The method 400b may include administering a progressed dose to the patient (block 420). The method 400b may include waiting for twenty to fifty-two hours (block 424). The method 400b may include determining whether the next progressed dosage is equal to the booster dosage (block 426). The booster dosage may be equal to a target dosage. If not, the next progressed dose may be administered to the patient (block 428). If so, the booster dose may be administered to the patient (block 430).

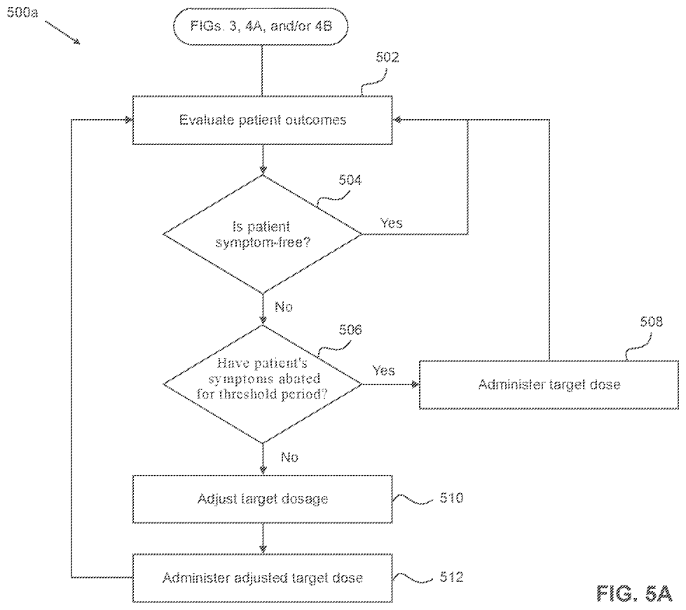

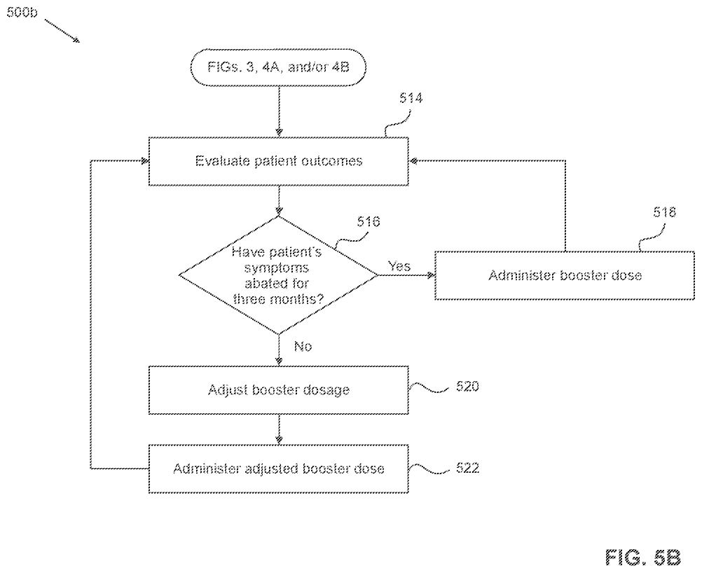

FIG. 5A illustrates a method 500a of evaluating an effectiveness of a target dosage for the psychoactive chemical, according to an embodiment. Some of the features in FIG. 5A may be the same as or similar to some of the features in the other FIGS. described herein as noted by same and/or similar reference characters, unless expressly described otherwise. Additionally, reference may be made to features shown in any of the other FIGS. described herein and not shown in FIG. 5A. Elements of the method 500a may be executed in one or more ways such as by a human, by a processing device, by mechanisms operating automatically or under human control, and so forth.

The method 500a may be a continuation of method 300, 400a, and/or 400b. The method 500a may include evaluating a patient's outcomes from previous treatments, including how long the patient has been symptom-free since the patient's last target dose (block 502). The method 500a may include determining whether the patient is experiencing symptoms of their mental health disorder (block 504). In one example, if the patient is symptom-free, a follow-up appointment may be scheduled to re-evaluate the patient's symptoms and outcomes at a later date. In another example, if the patient is symptom-free, a PHQ9 level of the patient may be monitored and subsequent follow-ups may be taken as needed. If the patient is not symptom-free, the method 500a may include determining whether the patient's symptoms have abated for a threshold period (block 506). If so, the method 500a may include administering the target dose to the patient (block 508). The target dose may be administered to the patient in one session. The target dose may be administered to the patient in one leg of a session, two legs of a session, three legs of a session, and so forth. If the patient's symptoms did not abate for the threshold period, the target dosage may be adjusted, such as increased (block 510). The method 500a may include administering the adjusted target dose (block 512). The patient may be scheduled for a follow-up visit where the patient's outcomes may again be evaluated.

FIG. 5B illustrates a method 500b of evaluating an effectiveness of a ketamine dosage, according to an embodiment. Some of the features in FIG. 5B may be the same as or similar to some of the features in the other FIGS. described herein as noted by same and/or similar reference characters, unless expressly described otherwise. Additionally, reference may be made to features shown in any of the other FIGS. described herein and not shown in FIG. 5B. Elements of the method 500b may be executed in one or more ways such as by a human, by a processing device, by mechanisms operating automatically or under human control, and so forth.

The method 500b may be a continuation of method 300, 400a, and/or 400b. The method 500b may include evaluating a patient's outcomes from previous treatments, including how long the patient has been symptom-free since the patient's last booster dose of ketamine (block 514). The method 500b may include determining whether the patient's symptoms have abated for three months (block 516). If so, the method 500b may include administering the booster dose of ketamine to the patient (block 518). If the patient's symptoms have not been abated for at least three months, the method 500b may include adjusting the booster dosage, such as by increasing the dosage amount (block 520). In one example, adjusting the booster dosage may include adding a leg to the patient's treatment. The method 500b may include administering the adjusted booster dose to the patient (block 522). The patient may be scheduled for a follow-up visit where the patient's outcomes may again be evaluated.

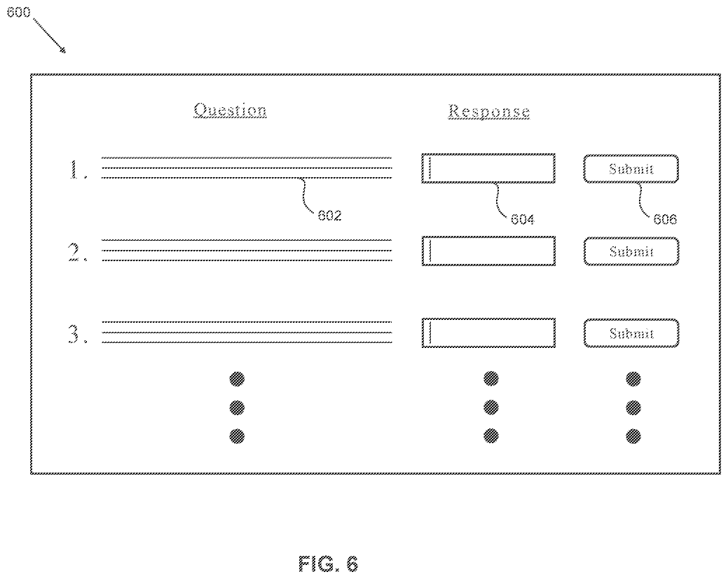

FIG. 6 illustrates a graphical user interface 600 for use in connection with evaluating an outcome of a patient in response to a treatment for a mental health disorder, according to an embodiment. The graphical user interface 600 can be executed by a processing device, such as any of the processing devices shown and described in connection with FIGS. 1 and 2 of this disclosure. The graphical user interface 600 can be displayed to a user (e.g., a patient) via an electronic display of the processing device. The graphical user interface 600 can allow a user to read text and to submit responses to queries for purposes or diagnosing or monitoring a mental health disorder.

The graphical user interface 600 can include a plurality of questions 602. For each question 602, the graphical user interface 600 can also display a corresponding response field 604. The user can enter a response by selecting a submit button 606. For example, selecting the submit button 606 (e.g., using a touch screen interface or pointing component of the processing device) can cause the processing device to store the data on a local storage device and/or to transmit the data to another processing device via a communication link such as any of the communication links 120 shown in FIG. 1.

The response fields 604 can be any type or form of graphical user interface element that allows the user to enter an input. For example, the response fields 604 may be text fields that allow the user to input any combination of letters or numbers. In some embodiments, there may be more than one response field 604 for a single question 602. For example, there may be five response fields 604 for a single question 602, and each response field 604 may correspond to an integer numerical value between one and 5. In embodiments in which multiple response fields 604 are present for a single questions, the response fields may be configured as radio buttons such that only a single one of the response fields 604 is selectable by the user at a given time. There may be more of fewer than five response fields for a single question 602.

The graphical user interface 600 can contain any number of questions 602. The user can enter a response for each question of the graphical user interface 600, and can submit each response using the corresponding submit buttons 606. Thus, a complete set of user responses to the questions can be received by the graphical user interface 600. The questions 602 can be questions of a standardized survey that may be used to probe for information from the patient relating to a presence or a severity of a mental health disorder. The responses entered by the user in each response field 604 can then be used, for example, to evaluate a condition of the patient, to diagnose the patient, or to determine whether the patient is a candidate for a particular therapy regimen, as described further below.

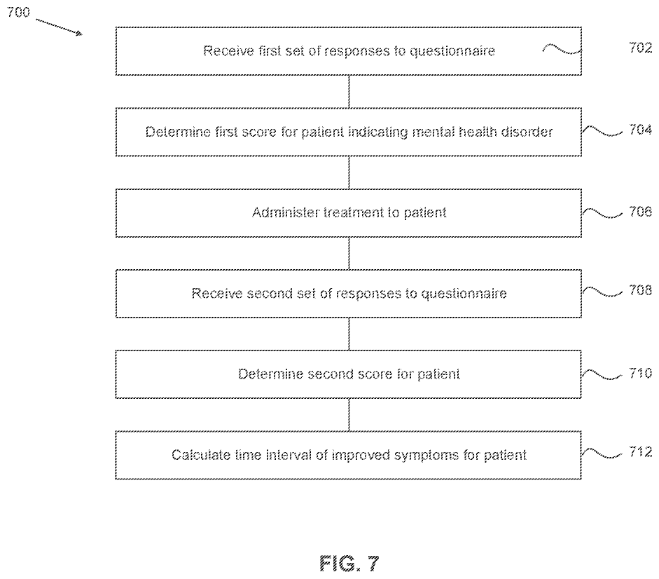

FIG. 7 illustrates a method 700 of evaluating an outcome of a patient in response to a treatment for a mental health disorder, according to an embodiment. In brief overview, the method 700 can include receiving a first set of responses from the patient to a health questionnaire (block 702). The method 700 can include determining a first score for the patient based on the first set of responses (block 704). The method 700 can include administering to the patient a treatment for the mental health disorder (block 706). The method 700 can include receiving a second set of responses from the patient to the health questionnaire after completion of the treatment (block 708). The method 700 can include determining a second score for the patient based on the second set of responses (block 710). The method can include calculating a time interval during which the patient remained free from the mental health disorder (block 712).

Referring again to FIG. 7, and in greater detail, the method 700 can include receiving a first set of responses from the patient to a health questionnaire (block 702). In some embodiments, the first set of responses can be received by a graphical user interface executing on a processing device. For example, the graphical user interface can be similar to or the same as the graphical user interface 600 shown in FIG. 6. The processing device can be any processing device capable of displaying a graphical user interface and receiving responses via the graphical user interfaces. The processing device can be any of the processing devices shown and described in connection with FIGS. 1 and 2. The health questionnaire can be a survey or series of questions that are relevant to a mental health condition of the patient, such as depression. The questions of the health questionnaire can be selected to probe for information related to a presence and/or a severity of symptoms associated with the mental health condition that the patient is experiencing. In some embodiments, the health questionnaire can be the Patient Health Questionnaire (PHQ)-9. In one example, the PHQ-9 may be collected on a daily basis, weekly basis, bi-weekly basis, monthly basis, and so forth.

The method 700 can include determining a first score for the patient based on the first set of responses (block 704). In some embodiments, this can be performed by a processing device coupled with the processing device that executes the graphical user interface used to receive the first set of responses in block 702. For example, the processing device can be included within the processing device processing device that executes the graphical user interface. In some other embodiments, the processor can be included within a separate device that is coupled with the processing device, for example via a computer network such as any of the communication links 120 shown in FIG. 1. In some embodiments, the patient's responses may be presented as numerical values corresponding to each question of the health questionnaire. Determining the first score can include analyzing, manipulating, or applying an algorithm to the numerical values to obtain the first score. For example, the processing device can determine the first score by summing the numerical values and identifying the sum as the first score. The processing device can compare the first score to a threshold score that may be indicative of the mental health disorder. Thus, if the first score exceeds the threshold, the processing device can determine that the patient is suffering from at least some symptoms of the mental health disorder. As a result, the patient may be a candidate for a treatment for the mental health disorder. The first score can serve as a baseline metric for evaluating a presence and/or a severity of the mental health disorder in the patient prior to any treatment. In this disclosure, this metric may be referred to as R1.

The method 700 can include administering to the patient a treatment for the mental health disorder (block 706). In some embodiments, the treatment may be or may include any of the treatments discussed in this disclosure, such as the treatments described in connections with FIG. 1, 2A, or 2B. For example, the treatment can include a dosage progression of a psychoactive chemical. The psychoactive chemical can include ketamine, which may be administered to the patient via any combination of an intramuscular injection, a nasal spray, or a ketamine troche. The dosage progression can include a diminished dosage, one or more progressed dosages, and a target dosage. Each of the dosages of the dosage progression can be administered to the patient during one or more therapy sessions.

The method 700 can include receiving a second set of responses from the patient to the health questionnaire after completion of the treatment (block 708). In some embodiments, this can be performed by a graphical user interface that is the same as or similar to the graphical user interface described above in connection with block 702. For example, the graphical user interface can be the graphical user interface 600 shown in FIG. 6. In some embodiments, the health questionnaire presented to the patient after completion of the treatment can be the same as the health questionnaire presented to the patient prior to treatment. Using the same health questionnaire can provide a consistent way to measure the patient's symptoms, thereby allowing for comparisons to be made between the patient's responses over time to evaluate changes in the patient's mental health condition.

The method 700 can include determining a second score for the patient based on the second set of responses (block 710). The second score can be determined in a manner that is similar to or the same as the manner used to determine the first score (block 704). In some embodiments, a processing device can determine the second score as described above in connection with block 704. The second score can be compared to the threshold value (e.g, by the processing device) indicating the mental health disorder of the patient. If the second score is lower than the threshold, it can be determined that the patient's symptoms have improved. For example, the patient may be free from the mental health disorder, indicating that the treatment has been effective.

The method can include calculating a time interval during which the patient remained free from the mental health disorder (block 712). In some embodiments, the time interval can be calculated by a processing device similar to or the same as the processing devices described above in connection with determining the first score (block 704) and determining the second score (block 710). In some embodiments, calculating the time interval can include periodically receiving additional sets of responses from the patient to the health questionnaire. For example, the patient may complete the health questionnaire daily, weekly, or monthly following completion of the treatment. In some embodiments, the patient may complete the health questionnaire at irregular intervals following completion of the treatment. When an additional set of responses to the health questionnaire is received after the patient has been treated, the method 700 can include determining respective additional scores corresponding to each of the additional sets of responses. Each score can be compared to the threshold score to determine whether, at the time the patient responded to the health questionnaire, the patient was still free from the mental health disorder. As a result, a physician or other healthcare practitioner can use the additional scores received from the patient after completion of the treatment to monitor the patient's condition on an ongoing basis. An interval end time at which a respective one of the additional scores exceeds the threshold can then be identified, which may indicate that the patient's symptoms have worsened or returned. The time interval can then be determined based on a time of completion of the treatment and the interval end time. This time interval can serve as a metric for evaluating the patient's immediate or short-term response to the treatment. In this disclosure, this time interval can be referred to as R3.

In some embodiments, the method 700 can include comparing the time interval (i.e., R3) to a predetermined reference interval. For example, the reference interval may be a period of one week, two weeks, three weeks, or one month. The reference interval may be two months, three months, or longer. Such a comparison can be performed, for example, by a processing device. The processing device can be the same processing device used to determine the first score (block 704) and the second score (block 710), and to calculate the time interval (block 712). In other implementations, a different processing device can perform the comparison. The method 700 can also include identifying the treatment as successful based on a determination that the time interval meets or exceeds the predetermined reference interval. For example, a treatment that results in only a short-term improvement for the patient, such as an improvement lasting less than the reference interval, may be classified as unsuccessful. If a treatment is unsuccessful, the method 700 can include determining an updated treatment for the patient. For example, the updated treatment may include higher dosages or more total dosages than the original treatment. The updated treatment can then be evaluated by repeating steps 706-712 of the method 700.

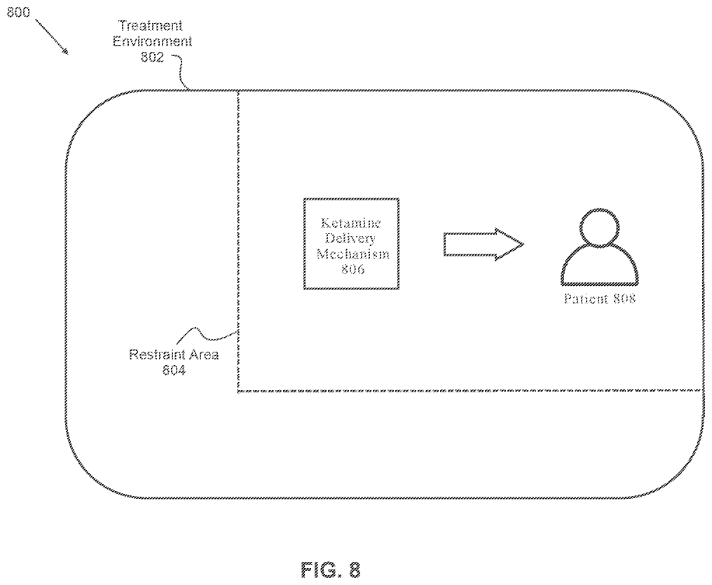

FIG. 8 illustrates a block diagram of a treatment environment 800 for treating a patient 806 having a mental health disorder using ketamine, according to an embodiment. The treatment environment can be or can include a room or a series of joined rooms. The treatment environment 800 can be configured to provide a comfortable and/or safe environment for the patient 806. For example, the treatment environment 800 can be free from sharp corners or hard surfaces.

In embodiments in which the treatment environment 800 includes corners, such corners can be modified to improve patient safety. For example, the treatment environment 800 can include a room having walls that meet at corners which could pose a danger to the patient if the patient experiences an emergence event, dizziness, or another condition impacting mobility or coordination of the patient 806 during the treatment. Such corners can be covered with a soft padding material. Padded material positioned near the sharp corners can help to reduce a risk of injury of the patient 806 upon impact with the corners. Similarly, a floor surface of the treatment environment 800 can be selected to improve patient safety in the event of a fall. For example, the floor surface of the treatment environment 800 can be carpeted or formed from a soft surface rather than a hard surface.

The treatment environment 800 can be a serene and peaceful setting to promote patient comfort and relaxation during treatment. The setting may imitate a natural setting (e.g. specific ecological environment) in color, decor, lighting, ambient sound, and so forth. A treatment ally or chaperone may be present in the setting to help the patient 806 navigate the feelings and emotions the patient 806 experiences during an active period after the patient receives a dose.

The treatment environment 800 can include a patient restraint area 802 positioned within the treatment environment 800 and configured to limit physical movement of the patient 806 during treatment. In some embodiments, the patient restraint area 802 can be defined by furniture items positioned within the treatment environment 800. For example, furniture items such as chairs, couches, and tables may be positioned to define the patient restraint area 802 and to prevent the patient 806 from moving outside of the patient restraint area 802 during the treatment. The patient restraint area 802 can be configured to respect a sense of freedom of the patient 806 and to not unduly limit the patient's motion during the treatment. For example, while inside the patient restraint area 802, the patient 806 may still be able to sit, stand, and walk freely within the patient restraint area 802.

The treatment environment 800 can include a ketamine delivery mechanism 804 for administering a ketamine dosage progression to the patient 806. The ketamine dosage progression can be any of the ketamine dosage progressions described in this disclosure. The ketamine dosage progression can include a diminished dosage, one or more progressed dosages, and a target dosage. Each dosage of the dosage progression can be administered via the ketamine delivery mechanism 804 to the patient 806 in the treatment environment 800 during one or more therapy sessions. In some embodiments, the ketamine delivery mechanism 804 can include at least one of a syringe for intramuscular injection of the ketamine, a nasal spray containing the ketamine, or a ketamine troche.

One innovative aspect of the subject matter described in this disclosure can be implemented in a method of treating a mental health disorder of a patient with ketamine. The method can include determining a protocol dosage for treating the mental health disorder of the patient with ketamine. The method can include determining a diminished dosage for treating the mental health disorder with the ketamine. The method can include determining a dosage progression. The dosage progression can include the diminished dosage, a first progressed dosage, a second progressed dosage, a third progressed dosage, a fourth progressed dosage, a fifth progressed dosage, a sixth progressed dosage, a seventh progressed dosage, an eighth progressed dosage, a ninth progressed dosage, and a target dosage. The method can include performing a first therapy session. The first therapy session can include administering the diminished dosage of the ketamine, waiting for a first inter-dosage period, and administering the first progressed dosage of the ketamine at the end of the first inter-dosage period. The method can include waiting for a first inter-session period. The method can include performing a second therapy session. The second therapy session can include administering the second progressed dosage of the ketamine, waiting for a second inter-dosage period, and administering the third progressed dosage of the ketamine at the end of the second inter-dosage period. The method can include waiting for a second inter-session period. The method can include performing a third therapy session. The third therapy session can include administering the fourth progressed dosage of the ketamine, waiting for a third inter-dosage period, and administering the fifth progressed dosage of the ketamine at the end of the third inter-dosage period. The method can include waiting for a third inter-session period. The method can include performing a fourth therapy session. The fourth therapy session can include administering the sixth progressed dosage of the ketamine, waiting for a fourth inter-dosage period, and administering the seventh progressed dosage of the ketamine at the end of the fourth inter-dosage period. The method can include waiting for a fourth inter-session period. The method can include performing a fifth therapy session including administering the eighth progressed dosage of the ketamine, waiting for a fifth inter-dosage period, and administering the ninth progressed dosage of the ketamine at the end of the fifth inter-dosage period. The method can include waiting for a fifth inter-session period. The method can include performing a sixth therapy session including administering three of the target dosages of the ketamine. Administration of each target dosage can be separated by a sixth inter-dosage period. In some embodiments, the method can include administering to the patient at least one of an anti-nauseant medication, an anti-hypertensive medication, and an anti-anxiety medication.

In some embodiments, the method can include determining a body mass of the patient and determining the target dosage to be 1.2 mg per kg of body mass of the patient. In some embodiments, determining the diminished dosage can include selecting the diminished dosage to be less than 0.5 mg per kg of body mass of the patient.

In some embodiments, the method can include determining that a body weight of the patient is less than 140 pounds. Determining the dosage progression can then include selecting each successive progressed dosage to be 5 mg greater than the previous progressed dosage, based on the determination that the body weight of the patient is less than 140 pounds.

In some embodiments, the method can include determining that a body weight of the patient is greater than or equal to 140 pounds. Determining the dosage progression can then include selecting each successive progressed dosage to be 10 mg greater than the previous progressed dosage, based on the determination that the body weight of the patient is greater than or equal to 140 pounds.

In some embodiments, a duration of each of the first inter-dosage period, the second inter-dosage period, the third inter-dosage period, the fourth inter-dosage period, the fifth inter-dosage period, and the sixth inter-dosage period can be selected based on a half-life of the ketamine. In some embodiments, each of the first inter-session period, the second inter-session period, the third inter-session period, the fourth inter-session period, and the fifth inter-session period can be of equal duration.

In some embodiments, the method can include waiting for a threshold period during which the patient's symptoms are improved after the sixth therapy session. The method can also include, in response to the patient's symptoms returning after the threshold period, performing a seventh therapy session including administering three booster dosages of the ketamine, each booster dosage separated by a seventh inter-dosage period. In some embodiments, an amount of each booster dosage is equal to the target dosage of the ketamine. In some embodiments, the method can include administering each dosage of the dosage progression using at least one of an intramuscular injection of the ketamine, a nasal spray containing the ketamine, or a ketamine troche.

Another inventive aspect of the subject matter described in this disclosure is directed to in a method of evaluating an outcome of a patient in response to a treatment for a mental health disorder. The method can include receiving, by a graphical user interface executing on a processing device, a first set of responses from the patient to a health questionnaire. The method can include determining, by a processing device communicatively coupled with the processing device, a first score for the patient based on the first set of responses. The first score can exceed a threshold indicating the mental health disorder of the patient. The method can include administering to the patient a treatment for the mental health disorder. The treatment can include a dosage progression of a psychoactive chemical including a diminished dosage, one or more progressed dosages, and a target dosage each administered to the patient during one or more therapy sessions. The method can include receiving, by the graphical user interface, a second set of responses from the patient to the health questionnaire after completion of the treatment. The method can include determining, by the processing device, a second score for the patient based on the second set of responses. The second score can be lower than the threshold, which may indicate that the patient's symptoms have improved. For example, the patient may be free from the mental health disorder. The method can include calculating, by the processing device, a time interval during which the patient remained free from the mental health disorder.

In some embodiments, the psychoactive chemical can include ketamine. In some embodiments, the method can further include determining the target dosage of the psychoactive chemical based on a body mass of the patient.

In some embodiments, calculating the time interval can include periodically receiving, by the graphical user interface, additional sets of responses from the patient to the health questionnaire. The method can include determining, by the processing device, respective additional scores corresponding to each of the additional sets of responses. The method can include identifying, by the processing device, an interval end time at which a respective one of the additional scores exceeds the threshold. The method can include determining, by the processing device, the time interval based on a time of completion of the treatment and the interval end time.

In some embodiments, the method can include comparing, by the processing device, the time interval to a predetermined reference interval. The method can also include identifying, by the processing device, the treatment as successful based on a determination that the time interval meets or exceeds the predetermined reference interval.

Another inventive aspect of the subject matter described in this disclosure is directed to a system for treating a patient having a mental health disorder using ketamine. The system can include a treatment environment for treating the patient. The system can include a patient restraint area positioned within the treatment environment and configured to limit physical movement of the patient during treatment. The system can include a ketamine delivery mechanism for administering a ketamine dosage progression to the patient. The ketamine dosage progression can include a diminished dosage, one or more progressed dosages, and a target dosage each administered to the patient in the treatment environment during one or more therapy sessions. The diminished dosage can be less than 0.5 mg per kg of body mass of the patient, and the target dosage can be 1.2 mg per kg of body mass of the patient. Other values can also be selected for the diminished dosage and/or the target dosage.

In some embodiments, the treatment environment can also include a room having walls that meet at corners. One or more padded surfaces can be positioned at the corners and configured to reduce a risk of injury to the patient upon impact with the corners. In some embodiments, the treatment environment can include a carpeted floor surface. In some embodiments, the ketamine delivery mechanism can include at least one of a syringe for intramuscular injection of the ketamine, a nasal spray containing the ketamine, a ketamine troche, or an embed or implanted administering device.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or embodiments described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D; and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader, narrower or equal in scope to the original claims, are to be considered within the subject matter of the examples described herein.

1. A method, comprising:
determining a protocol dosage for treating a mental health disorder of a patient with ketamine;
determining a diminished dosage for treating the mental health disorder with the ketamine;
determining a dosage progression comprising the diminished dosage, a first progressed dosage, a second progressed dosage, a third progressed dosage, a fourth progressed dosage, a fifth progressed dosage, a sixth progressed dosage, a seventh progressed dosage, an eighth progressed dosage, a ninth progressed dosage, and a target dosage;
performing a first therapy session comprising:
administering the diminished dosage of the ketamine;
waiting for a first inter-dosage period; and
administering the first progressed dosage of the ketamine at the end of the first inter-dosage period;
waiting for a first inter-session period;
performing a second therapy session comprising:
administering the second progressed dosage of the ketamine;
waiting for a second inter-dosage period; and
administering the third progressed dosage of the ketamine at the end of the second inter-dosage period;
waiting for a second inter-session period;
performing a third therapy session comprising:
administering the fourth progressed dosage of the ketamine;
waiting for a third inter-dosage period; and
administering the fifth progressed dosage of the ketamine at the end of the third inter-dosage period;
waiting for a third inter-session period;
performing a fourth therapy session comprising:
administering the sixth progressed dosage of the ketamine;
waiting for a fourth inter-dosage period; and
administering the seventh progressed dosage of the ketamine at the end of the fourth inter-dosage period;
waiting for a fourth inter-session period;
performing a fifth therapy session comprising:
administering the eighth progressed dosage of the ketamine;
waiting for a fifth inter-dosage period; and
administering the ninth progressed dosage of the ketamine at the end of the fifth inter-dosage period;
waiting for a fifth inter-session period; and
performing a sixth therapy session comprising administering three of the target dosages of the ketamine, each target dosage separated by a sixth inter-dosage period.

2. The method of claim 1, further comprising administering to the patient at least one of an anti-nauseant medication, an anti-hypertensive medication, or an anti-anxiety medication.

3. The method of claim 1, further comprising:
determining a body mass of the patient; and
determining the target dosage to be 1.2 milligrams (mg) per kilogram (kg) of body mass of the patient.

4. The method of claim 1, wherein determining the diminished dosage comprises selecting the diminished dosage to be less than 0.5 mg per kg of body mass of the patient.

5. The method of claim 1, further comprising:
determining that a body weight of the patient is less than 140 pounds, wherein determining the dosage progression comprises selecting each successive progressed dosage to be 5 mg greater than the previous progressed dosage, based on the determination that the body weight of the patient is less than 140 pounds.

6. The method of claim 1, further comprising:
determining that a body weight of the patient is greater than or equal to 140 pounds, wherein determining the dosage progression comprises selecting each successive progressed dosage to be 10 mg greater than the previous progressed dosage, based on the determination that the body weight of the patient is greater than or equal to 140 pounds.

7. The method of claim 1, wherein a duration of each of the first inter-dosage period, the second inter-dosage period, the third inter-dosage period, the fourth inter-dosage period, the fifth inter-dosage period, and the sixth inter-dosage period is selected based on a half-life of the ketamine.

8. The method of claim 1, wherein each of the first inter-session period, the second inter-session period, the third inter-session period, the fourth inter-session period, and the fifth inter-session period are of equal duration.

9. The method of claim 1, further comprising:
waiting for a threshold period during which the patient's symptoms are improved after the sixth therapy session; and
in response to the patient's symptoms returning after the threshold period, performing a seventh therapy session comprising administering three booster dosages of the ketamine, each booster dosage separated by a seventh inter-dosage period.

10. The method of claim 9, wherein an amount of each booster dosage is equal to the target dosage of the ketamine.

11. The method of claim 1, further comprising administering each dosage of the dosage progression using at least one of an intramuscular injection of the ketamine, a nasal spray containing the ketamine, or a ketamine troche.

12. A method, comprising:
receiving, by a graphical user interface executing on a processing device, a first set of responses from a patient to a health questionnaire;
determining, by a processing device communicatively coupled with the processing device, a first score for the patient based on the first set of responses, wherein the first score exceeds a threshold indicating a mental health disorder of the patient;
administering to the patient a treatment for the mental health disorder, the treatment comprising a dosage progression of a psychoactive chemical including a diminished dosage, one or more progressed dosages, and a target dosage each administered to the patient during one or more therapy sessions;
receiving, by the graphical user interface, a second set of responses from the patient to the health questionnaire after completion of the treatment;
determining, by the processing device, a second score for the patient based on the second set of responses, wherein the second score is lower than the threshold;
calculating, by the processing device, a time interval during which the patient remained free from the mental health disorder.

13. The method of claim 12, wherein the psychoactive chemical comprises ketamine.

14. The method of claim 12, further comprising determining the target dosage of the psychoactive chemical based on a body mass of the patient.

15. The method of claim 12, wherein calculating the time interval further comprises:
periodically receiving, by the graphical user interface, additional sets of responses from the patient to the health questionnaire;
determining, by the processing device, respective additional scores corresponding to each of the additional sets of responses;
identifying, by the processing device, an interval end time at which a respective one of the additional scores exceeds the threshold; and
determining, by the processing device, the time interval based on a time of completion of the treatment and the interval end time.

16. The method of claim 15, further comprising:
comparing, by the processing device, the time interval to a predetermined reference interval; and
identifying, by the processing device, the treatment as successful based on a determination that the time interval meets or exceeds the predetermined reference interval.

17. A system, comprising:
a treatment environment for treating a patient having a mental health disorder using ketamine;
a patient restraint area positioned within the treatment environment, the patient restraint area configured to limit physical movement of the patient during treatment;
a ketamine delivery mechanism for administering a ketamine dosage progression to the patient, the ketamine dosage progression comprising:
a diminished dosage;
one or more progressed dosages; and
a target dosage each administered to the patient in the treatment environment during one or more therapy sessions, wherein:
the diminished dosage is less than 0.5 mg per kg of body mass of the patient; and
the target dosage is 1.2 mg per kg of body mass of the patient.

18. The system of claim 17, wherein the treatment environment comprises:
a room having walls that meet at corners; and
one or more padded surfaces positioned at the corners and configured to reduce a risk of injury to the patient upon impact with the corners.

19. The system of claim 17, wherein the treatment environment comprises a carpeted floor surface.

20. The system of claim 17, wherein the ketamine delivery mechanism comprises at least one of a syringe for intramuscular injection of the ketamine, a nasal spray containing the ketamine, or a ketamine troche.
